(19) United States
(12) Patent Application Publication
(10) Pub. No.: US 2014/0221469 A1
(43) Pub. Date: Aug. 7, 2014

(54) PHYTOCANNABINOIDS FOR USE IN THE TREATMENT OF CANCER

(71) Applicants: Ruth Alexandra Ross, Aberdeen (GB); Daniela Parolaro, Varese (IT)

(72) Inventors: Ruth Alexandra Ross, Aberdeen (GB); Daniela Parolaro, Varese (IT)

(73) Assignees: Otsuka Pharmaceutical Co., Ltd., Tokyo (JP); GW Pharma Limited, Salisbury, Wiltshire (GB)

(21) Appl. No.: 14/343,877

(22) PCT Filed: Sep. 10, 2012
(86) PCT No.: PCT/GB2012/052224

§ 371 Date: Mar. 10, 2014

(30) Foreign Application Priority Data
Sep. 12, 2011 (GB) 1115711.2

(51) Int. Cl. A61K 31/352; A61K 31/235; A61K 31/05

(52) U.S. Cl. CPC A61K 31/352; A61K 31/05; A61K 31/235
USPC 514/454; 549/390; 568/743; 562/469; 514/733; 514/544

(57) ABSTRACT

The present invention relates to the use of phytocannabinoids in the treatment of cancer. More particularly it relates to the use of phytocannabinoids in the treatment of tumour cell invasion and cell migration or metastases. Cancers, where invasion and cell migration plays a key role in prognosis include brain tumours, more particularly gliomas, and most particularly Glioblastoma multiforme (GBM) and breast cancers. The phytocannabinoids tetrahydrocannabivarin (THCV) and cannabidivarin (CBDV) alone or in combination with each other and/or other phytocannabinoids, particularly cannabidiol (CBD), tetrahydrocannabinol (THC) and cannabigerol (CBG) or their respective acids are of particular use.

The present invention relates to the use of phytocannabinoids in the treatment of cancer. More particularly it relates to the use of phytocannabinoids in the treatment of tumour cell invasion and cell migration or metastases.

Cancers, where invasion and cell migration plays a key role in prognosis include brain tumours, more particularly gliomas, and most particularly Glioblastoma multiforme (GBM) and breast cancers.

In a first embodiment the invention relates to the use of the phytocannabinoids tetrahydrocannabivarin (THCV) and cannabidivarin (CBDV) alone or in combination with each other and/or other phytocannabinoids, particularly cannabidiol (CBD), tetrahydrocannabinol (THC) and cannabigerol (CBG) or their respective acids in the treatment of glioma and other cancers which are invasive or have a tendency to migrate. This may be for the purpose of preventing invasion or migration as opposed to, or in addition to, preventing proliferation.

In a second embodiment the invention relates to the use of the phytocannabinoid tetrahydrocannabinol acid (THCA) or cannabidiolic acid (CBDA) in the treatment of breast cancer and other cancers which are invasive or have a tendency to migrate. This too may be for the purpose of preventing invasion or migration as opposed to, or in addition to, preventing proliferation.

### BACKGROUND TO THE INVENTION

Malignant gliomas are defined as the most deadly human brain tumours, with poor prognosis. A number of recent studies have suggested a potential use of compounds derived from marijuana as suppressors of tumour cell growth in gliomas.

Cannabinoids have been shown to have an anti-proliferative effect on different cancer cell lines. The cannabinoids THC, THCA, CBD, CBDA, CBG and CBC and the cannabinoid BDS THC and CBD were tested on eight different cell lines including DU-145 (hormone-sensitive prostate cancer), MDA-MB-231 (breast cancer), CaCo-2 (colorectal cancer) and C6 (glioma cells). (Ligresti, 2006).

The anti-proliferative effects of CBD have also been evaluated on U87 and U373 human glioma cell lines, (Massi, 2004). The anti-proliferative effect of CBD was correlated to induction of apoptosis, as determined by cytofluorimetric analysis and single-strand DNA staining, which was not reverted by cannabinoid antagonists. In addition CBD, administered subcutaneously to nude mice at the dose of 0.5 mg/mouse, significantly inhibited the growth of subcutaneously implanted U87 human glioma cells. It was concluded that CBD was able to produce a significant anti-tumour activity both in vitro and in vivo, thus suggesting a possible application of CBD as a chemotherapeutic agent.

The application WO 2006/037981 describes the use of the cannabinoid CBD to prevent tumour cells migrating or metastasising from an area of uncontrolled growth to an area away from the original tumour site. CBD caused a concentration-dependent inhibition of the migration of U87 glioma cells, quantified in a Boyden chamber. Since these cells express both cannabinoid CB1 and CB2 receptors in the membrane, the group also evaluated their engagement in the anti-migratory effect of CBD.

Cannabinoids have been shown to play a fundamental role in the control of cell survival/cell death. It has been reported that cannabinoids may induce proliferation, growth arrest, or apoptosis in a number of cells, including neurons, lymphocytes, and various transformed neural and non-neural cells, and that cannabinoids induce apoptosis of glioma cells in culture and regression of malignant gliomas in vivo (Guzman, 2001).

A pilot clinical study of THC in patients with recurrent glioblastoma multiforme has been conducted. This pilot phase I trial consisted of nine patients with recurrent glioblastoma multiforme who were administered THC intra-tumourally. The patients had previously failed standard therapy (surgery and radiotherapy) and had clear evidence of tumour progression. The primary end point of the study was to determine the safety of intracranial THC administration. They also evaluated THC action on the length of survival and various tumour-cell parameters. Median survival of the cohort from the beginning of cannabinoid administration was 24 weeks (95% confidence interval: 15-33).

The application WO 2008/144475 describes treating cell proliferation disorders including cancer with cannabidiol derivatives either alone or in combination with THC or a derivative thereof.

The application WO 2009/147439 describes the use of a combination of cannabinoids, particularly tetrahydrocannabinol (THC) and cannabidiol (CBD), in the manufacture of a medicament for use in the treatment of cancer. In particular the cancer to be treated is a brain tumour, more particularly a glioma; more particularly still a glioblastoma multiforme (GBM).

The application WO 2009/147438 describes the use of one or more cannabinoids, particularly THC and/or CBD in combination with a non-cannabinoid chemotherapeutic agent in the manufacture of a medicament for use in the treatment of cancer. In particular the cancer to be treated is a brain tumour, more particularly a glioma, more particularly still a glioblastoma multiforme (GBM). The non-cannabinoid chemotherapeutic agent may be a selective estrogen receptor modulator or an alkylating agent.

Galanti et al. (2008) describes the use of THC to inhibit cell cycle progression in human glioblastoma multiforme cells and discusses the mechanism whereby this cannabinoid is thought to work by.

De Petrocellis et al. (2010) describe the effects of cannabinoids and cannabis extracts on TRP channels. There is discussion that the activity of these cannabinoids at particular TRP channels may be beneficial in the treatment of different cancers.

The application GB 2448535 also describes the activity of different cannabinoids at different TRP channels. The application describes five different cancers; of which glioma is one and eight different cannabinoids.

### DEFINITIONS AND ABBREVIATIONS

Definitions of some of the terms used to describe the invention are detailed below:

The main phytocannabinoids described in the present application are listed below along with their standard abbreviations.

| | | |
|---|---|---|
| CBD | Cannabidiol | 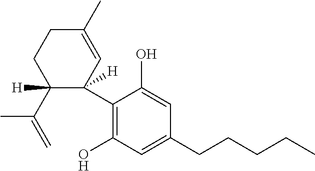 [SMILES: C=C(C)[C@@H]1CCC(C)=C[C@H]1c1c(O)cc(CCCCC)cc1O] |
| CBDA | Cannabidiolic acid | 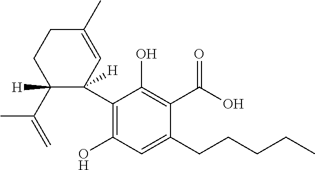 [SMILES: C=C(C)[C@@H]1CCC(C)=C[C@H]1c1c(O)cc(CCCCC)c(C(=O)O)c1O] |
| CBDV | Cannabidivarin | 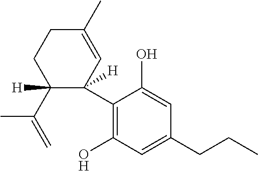 [SMILES: C=C(C)[C@@H]1CCC(C)=C[C@H]1c1c(O)cc(CCC)cc1O] |
| CBDVA | Cannabidivarinic acid | 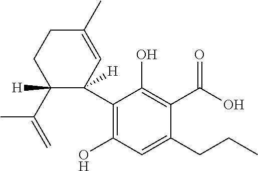 [SMILES: C=C(C)[C@@H]1CCC(C)=C[C@H]1c1c(O)cc(CCC)c(C(=O)O)c1O] |
| THC | Tetrahydrocannabinol | 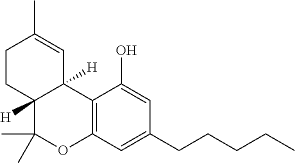 [SMILES: CCCCCc1cc(O)c2c(c1)OC(C)(C)[C@@H]1CCC(C)=C[C@@H]21] |
| THCA | Tetrahydrocannabinolic acid | 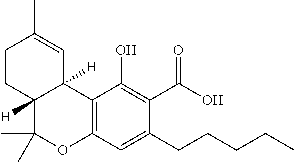 [SMILES: CCCCCc1cc2c(c(O)c1C(=O)O)[C@@H]1C=C(C)CC[C@H]1C(C)(C)O2] |
| THCV | Tetrahydrocannabivarin | 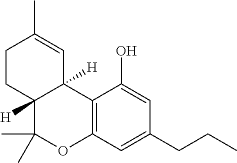 [SMILES: CCCc1cc(O)c2c(c1)OC(C)(C)[C@@H]1CCC(C)=C[C@@H]21] |
| THCVA | Tetrahydrocannabivarinic acid | 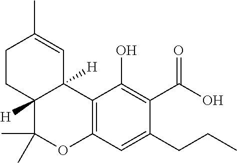 [SMILES: CCCc1cc2c(c(O)c1C(=O)O)[C@@H]1C=C(C)CC[C@H]1C(C)(C)O2] |
| CBG | Cannabigerol | 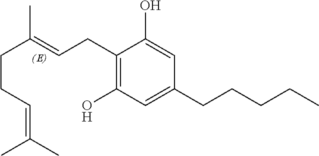 [SMILES: CCCCCc1cc(O)c(C/C=C(\C)CCC=C(C)C)c(O)c1] |

The table above is not exhaustive and merely details the cannabinoids which are identified in the present application for reference. So far over 60 different cannabinoids have been identified and these cannabinoids can be split into different groups as follows: Phytocannabinoids; Endocannabinoids and Synthetic cannabinoids.

“Phytocannabinoids” are cannabinoids that originate from nature and can be found in the cannabis plant. The phytocannabinoids can be isolated or present as a botanical drug substance or be produced synthetically.

An “isolated cannabinoid” is defined as a phytocannabinoid that has been extracted from the cannabis plant and purified to such an extent that all the additional components, such as secondary and minor cannabinoids and the non-cannabinoid fraction have been removed.

A “botanical drug substance” or “BDS” is defined in the Guidance for Industry Botanical Drug Products Draft Guidance, August 2000, US Department of Health and Human Services, Food and Drug Administration Centre for Drug Evaluation and Research as: “A drug derived from one or more plants, algae, or microscopic fungi. It is prepared from botanical raw materials by one or more of the following processes: pulverisation, decoction, expression, aqueous extraction, ethanolic extraction or other similar processes.” A botanical drug substance does not include a highly purified or chemically modified substance derived from natural sources. Thus, in the case of cannabis, BDS derived from cannabis plants do not include highly purified Pharmacopoeial grade cannabinoids.

Phytocannabinoids can be found as either the neutral (decarboxylated form) or the carboxylic acid form depending on the method used to extract the cannabinoids. For example, it is known that heating the carboxylic acid form will cause most of the carboxylic acid form to decarboxylate into the neutral form.

Phytocannabinoids can also occur as either the pentyl (5 carbon atoms) or propyl (3 carbon atoms) variant. Initially it was thought that the propyl and pentyl variants would have similar properties, however recent research has found that this may not be true. For example the phytocannabinoid THC is known to be a CB1 receptor agonist whereas the propyl variant THCV has been discovered to be a CB1 receptor antagonist meaning that it has almost opposite effects.

In the present invention a BDS is considered to have two components: the phytocannabinoid-containing component and the non-phytocannabinoid containing component. Preferably the phytocannabinoid-containing component is the larger component comprising greater than 50% (w/w) of the total BDS and the non-phytocannabinoid containing component is the smaller component comprising less than 50% (w/w) of the total BDS.

The amount of phytocannabinoid-containing component in the BDS may be greater than 55%, through 60%, 65%, 70%, 75%, 80% to 85% or more of the total extract. The actual amount is likely to depend on the starting material used and the method of extraction used.

The “principle phytocannabinoid” in a BDS is the phytocannabinoid that is present in an amount that is higher than that of the other phytocannabinoids. Preferably the principle phytocannabinoid is present in an amount greater than 40% (w/w) of the total extract. More preferably the principle phytocannabinoid is present in an amount greater than 50% (w/w) of the total extract. More preferably still the principle phytocannabinoid is present in an amount greater than 60% (w/w) of the total extract.

The amount of the principle phytocannabinoid in the BDS is preferably greater than 75% of the phytocannabinoid-containing fraction, more preferably still greater than 85% of the phytocannabinoid-containing fraction, and more preferably still greater than 95% of the phytocannabinoid-containing fraction.

In some cases, such as where the principle cannabinoid is either CBDV or THCVA the amount of the principle phytocannabinoid in the BDS is lower. Here the amount of phytocannabinoid is preferably greater than 55% of the phytocannabinoid-containing fraction.

The “secondary phytocannabinoid/s” in a BDS is the phytocannabinoid/s that is/are present in significant proportions. Preferably the secondary phytocannabinoid is present in an amount greater than 5% (w/w) of the total extract, more preferably greater than 10% (w/w) of the total extract, more preferably still greater than 15% (w/w) of the total extract. Some BDS's will have two or more secondary phytocannabinoids that are present in significant amounts. However not all BDS's will have a secondary phytocannabinoid. For example CBG BDS does not have a secondary phytocannabinoid in its extract.

The “minor phytocannabinoid/s” in a BDS can be described as the remainder of all the phytocannabinoid components once the principle and secondary phytocannabinoids are accounted for. Preferably the minor phytocannabinoids are present in total in an amount of less than 10% (w/w) of the total extract, more preferably still less than 5% (w/w) of the total extract, and most preferably the minor phytocannabinoid is present in an amount less than 2% (w/w) of the total extract.

Typically the non-phytocannabinoid containing component of the BDS comprises terpenes, sterols, triglycerides, alkanes, squalenes, tocopherols and carotenoids.

These compounds may play an important role in the pharmacology of the BDS either alone or in combination with the phytocannabinoid.

The “terpene fraction” may be of significance and can be broken down by the type of terpene: monoterpene or sesquiterpene. These terpene components can be further defined in a similar manner to the cannabinoids.

The amount of non-phytocannabinoid containing component in the BDS may be less than 45%, through 40%, 35%, 30%, 25%, 20% to 15% or less of the total extract. The actual amount is likely to depend on the starting material used and the method of extraction used.

The “principle monoterpene/s” in a BDS is the monoterpene that is present in an amount that is higher than that of the other monoterpenes. Preferably the principle monoterpene/s is present in an amount greater than 20% (w/w) of the total terpene content. More preferably the principle monoterpene is present in an amount greater than 30% (w/w) of the total terpene content, more preferably still greater than 40% (w/w) of the total terpene content, and more preferably still greater than 50% (w/w) of the total terpene content. The principle monoterpene is preferably a myrcene or pinene. In some cases there may be two principle monoterpenes. Where this is the case the principle monoterpenes are preferably a pinene and/or a myrcene.

The “principle sesquiterpene” in a BDS is the sesquiterpene that is present in an amount that is higher than all the other terpenes. Preferably the principle sesquiterpene is present in an amount greater than 20% (w/w) of the total terpene content; more preferably still t greater than 30% (w/w) of the total terpene content. The principle sesquiterpene is preferably a caryophyllene and/or a humulene.

The sesquiterpene components may have a “secondary sesquiterpene”. The secondary monoterpene is preferably a pinene, which is preferably present at an amount greater than 5% (w/w) of the total terpene content, more preferably the secondary terpene is present at an amount greater than 10% (w/w) of the total terpene content.

The secondary sesquiterpene is preferably a humulene which is preferably present at an amount greater than 5% (w/w) of the total terpene content, more preferably the secondary terpene is present at an amount greater than 10% (w/w) of the total terpene content.

Alternatively botanical extracts may be prepared by introducing isolated phytocannabinoids into a non-cannabinoid plant fraction as can be obtained from a zero cannabinoid plant or a CBG-free BDS.

It is an object of the present invention to identify alternative and potentially more effective treatments for gliomas and breast cancers than existing treatments and candidate phytocannabinoids such as THC and/or CBD.

### BRIEF SUMMARY OF THE DISCLOSURE

In accordance with a first aspect of the present invention there is provided THCV, CBDV, CBDA or THCA for use in the treatment of an invasive or migratory cancer.

In a preferred embodiment THCV or CBDV are used, though not exclusively, in the treatment of gliomas, particularly GBM.

The THCV or CBDV are preferably used for the purpose of preventing invasion or migration (or metastases).

The THCV or CBDV may be used in combination with one or more other cannabinoids, such as THC and/or CBD. The combination of THCV and CBD was found to be particularly beneficial.

The ratio of THCV to CBD may be in the range of 5:1 to 1:5, more preferably 3:1 to 1:3 and most preferably 2:1 to 1:2. The combination is anti-proliferative.

In another embodiment THCA or CBDA are used, though not exclusively, in the treatment of breast cancer.

The THCA or CBDA are preferably used for the purpose of preventing invasion or migration (or metastases).

The invention also extends to pharmaceutical compositions, methods of treatment and methods of manufacturing medicaments for use in the treatment of cancer.

### BRIEF DESCRIPTION OF THE DRAWINGS

Embodiments of the invention are further described hereinafter with reference to the accompanying drawings, in which:

FIG. 1 illustrates dose response curves for CBG, CBDV and THCV and demonstrates their anti-proliferative effect on a human glioma cell line (U87);

FIGS. 2A, 2B and 2C show the effect of a CB1 antagonist, a CB2 antagonist, and a TRPV1 antagonist on inhibition of cell proliferation induced by CBG;

FIGS. 3A, 3B and 3C show the effect of a CB1 antagonist, a CB2 antagonist, and a TRPV1 antagonist on inhibition of cell proliferation induced by CBDV;

FIGS. 4A, 4B and 4C show the effect of a CB1 antagonist, a CB2 antagonist, and a TRPV1 antagonist on inhibition of cell proliferation induced by THCV;

FIGS. 5A, 5B, 5C and 5D show the inhibition of proliferation of glioma cells on co-administration of CBD and THCV at different concentrations and ratios (approx. 2:1 to 1:2);

FIGS. 6A, 6B, and 6C show the degree of apoptosis of glioma cells on co-administration of CBD and THCV at different concentrations and ratios (approx. 2:1 to 1:2);

FIGS. 7A and 7B show the effect of THCV on cell migration and invasion respectively at different concentrations;

FIGS. 8A and 8B show the effect of CBDV on cell migration and invasion respectively at different concentrations;

FIG. 9 illustrates dose response curves for CBG, CBDV and THCV and demonstrates their anti-proliferative effect on a different human glioma cell line (T98G);

FIGS. 10A and 10B illustrate cell viability of glioma cells (T98G) in response to increasing concentrations of CBDV;

FIGS. 11A and 11B illustrate cell viability of glioma cells (T98G) in response to increasing concentrations of CBG;

FIGS. 12A and 12B illustrate cell viability of glioma cells (T98G) in response to increasing concentrations of THCV;

FIGS. 13A and 13B show the effect of a CB1 antagonist and a CB2 antagonist respectively on inhibition of cell proliferation induced by CBDV;

FIGS. 14A and 14B show the effect of a CB1 antagonist and a CB2 antagonist respectively on inhibition of cell proliferation induced by CBG;

FIGS. 15A and 15B show the effect of a CB1 antagonist and a CB2 antagonist respectively on inhibition of cell proliferation induced by THCV;

FIGS. 16A and 16B show the effect of CBDV on cell migration and invasion in T98G cells;

FIGS. 17A and 17B and 17C and 17D show the effect of CBG on cell migration and invasion in U87 and T98G cells respectively;

FIGS. 18A and 18B show the effect of THCV on cell migration and invasion in T98G cells; and

FIG. 19 shows the anti-migratory effect of CBDA and THCA on a human breast cell line MDA MB 231.

### DETAILED DESCRIPTION

The Examples below illustrate the activity of three phytocannabinoid, THCV, CBDV and CBG, in human glioma cell lines, and two phytocannabinoids, CBDVA and THCVA, in a human breast cell line. Depending on the phytocannabinoid up to four aspects were evaluated:
1. Cell viability;
2. Apoptosis;
3. Cell motility; and
4. Invasiveness.

In glioma, no comparative data is shown for THC and CBD since their effect in glioma is well documented.

Additionally, combination data showing the combined effect of THCV and CBD is given.

### Examples 1 to 4

### Activity of Phytocannabinoids in Glioma Cell Line U87-MG

### Materials and Methods

Reagents:

Standard chemicals and cell culture reagents were purchased from Sigma-Aldrich Srl (Italy). THCV, CBDV and CBG were natural phytocannabinoids isolated from cannabis. They were initially dissolved in ethanol to a concentration of 50 mM and stored at −20° C. and further diluted in complete tissue culture medium; final ethanol concentration never exceeded 0.05%.

Cell Culture:

The human glioma cell line U87-MG was obtained from the American Type Culture Collection (Rockville, USA). Cells were maintained in DMEM supplemented with 10% heat-inactivated foetal bovine serum (Euroclone, Italy), 1% glutamine, 1% antibiotic mixture, 1% sodium pyruvate, 1% non-essential amino acids, at 37° C. in a humidified 5% CO₂ atmosphere. Cells were seeded in complete medium. After a 24 h incubation, the medium was replaced by serum-free medium (ITSS medium), consisting of DMEM supplemented with 5 μg/ml insulin, 5 μg/ml transferrin, and 5 μg/ml sodium selenite.

### Example 1

### Analysis of Cell Viability

To determine the effects of the phytocannabinoids upon cell viability, the applicants employed a MTT colorimetric assay ([3-(4,5-dimethyl-2-thiazolyl)-2,5-diphenyl-2H tetrazolium bromide]; Sigma-Aldrich). Briefly, U87 human glioma cells were seeded in a 96-well flat bottom multi-well at a density of 8000 cells/well. After 24 h, cells were treated with THCV, CBDV and CBG and/or receptor antagonists at the indicated concentrations. At the end of the incubation with the drugs, MTT (0.5 mg/ml final concentration) was added to each well and the incubation was then continued for 4 h. The insoluble formazan crystals were solubilized by the addition of 100 μl of 100% dimethyl sulfoxide. The plates were read at 570 nm using an automatic microtiter plate reader.

### Example 2

### Evaluation of Apoptosis

3.4×10⁵ tumour cells were cultured in 6 well-plates in the presence or absence of CBDV or THCV for 24 h, as described above and the percentage of apoptotic cells on the total cell population (adhering/detached cells) was evaluated. Briefly, cells were collected, washed, and centrifuged at 1300 rpm. They were then fixed in ethanol 70% for at least 30 min at −20° C. After centrifugation, the cell pellet was gently re-suspended in 1 ml of PBS solution containing propidium iodide (PI, 50 μg/ml) and RNAse (20 μg/ml). Cells were analyzed after a minimum of 30 min of incubation in the dark at room temperature, and apoptosis was detected in individual cells using a flow cytometer (equipped with a single 488-nm argon laser; BD Biosciences, San Jose, Calif.) by reduced fluorescence of PI in apoptotic nuclei.

### Example 3

### Cell Motility Assay

BD BioCoat Control Inserts (BD, USA) were used to examine the ability of U87-MG cells to migrate through an 8.0 micron pore size PET membrane. U87-MG cells (2.5×10⁴ cells) were re-suspended in 500 μl of serum-free medium in presence of phytocannabinoids, and added to the upper chamber. The lower chamber was filled with 0.75 ml of complete medium as chemo-attractant. Cells were then incubated for 22 h at 37° C. After removal of cells on the upper surface of the membrane, cells on the lower surface were fixed in 100% methanol and stained with Diff-Quick stain (Medion Diagnostics, USA). Sixteen fields of cells were counted randomly in each well under a light microscope at 200× magnification. Data was expressed as the percentage of migrating cells as compared with the control. All the experiments were performed in triplicates and results were expressed as mean±SEM of three independent experiments.

### Example 4

### Cell Invasion Assay

BD BioCoat matrigel invasion chambers (BD, USA) were used to examine the ability of U87 cells to penetrate the extracellular matrix. U87 cells (2.5×10⁴ cells) were re-suspended in 500 μl of serum-free medium in the presence of the phytocannabinoids, and added to the upper chamber. The lower chamber was filled with 0.75 ml of complete medium as chemo-attractant. Cells were then incubated for 22 h at 37° C. After removal of cells on the upper surface of the membrane, cells on the lower surface were fixed in 100% methanol and stained with Diff-Quick stain (Medion Diagnostics, USA). Sixteen fields of cells were counted randomly in each well under a light microscope at 200× magnification. Data was expressed as the percentage of invasive cells as compared with the control. All the experiments were performed in triplicates and results were expressed as mean±SEM of three independent experiments.

### Results

Evaluation of the Anti-Proliferative Effect of Phytocannabinoids:

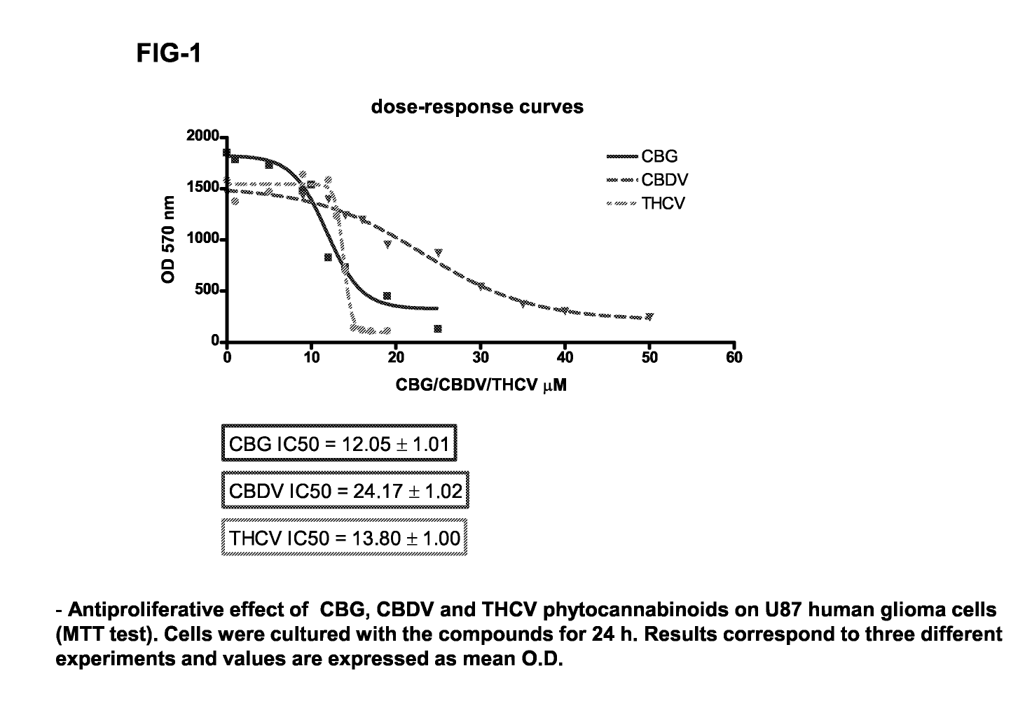

Cannabidivarin (CBDV), cannabigerol (CBG) and tetrahydrocannabivarin (THCV) all inhibited the growth of human U87 glioma cells. The addition of CBDV, CBG and THCV to the culture medium led to a dramatic drop of mitochondrial oxidative metabolism (MTT test), in a concentration-dependent manner, already evident 24 h after cannabinoid exposure with an IC₅₀ of 24.17, 12.05 and 13.80 μM, respectively (FIG. 1).

### Evaluation of the Involvement of Cannabinoid and Vanilloid Receptors in the Anti-Proliferative Effect of Phytocannabinoids

CBG:

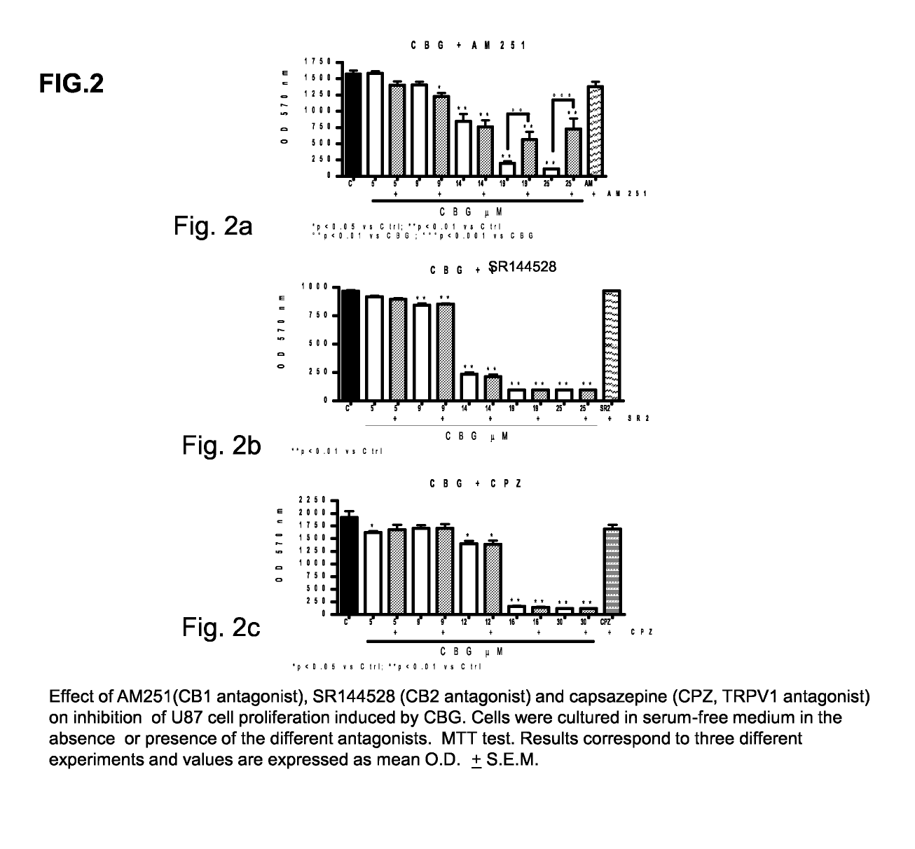

Further experiments aimed at clarifying the role of cannabinoid receptors in CBG-induced effects, showed that the CB1 cannabinoid antagonist AM 251 (0.5 μM) was able to antagonize the inhibitory action of the phytocannabinoid on glioma cells growth only at 19 and 25 μM concentrations (FIG. 2A). In contrast, either CB2 receptor antagonist SR 144528 (0.5 μM) or the vanilloid receptor antagonist capsazepine (0.625 μM) failed to antagonize the anti-proliferative effect of the compound (FIGS. 2B and 2C).

CBDV:

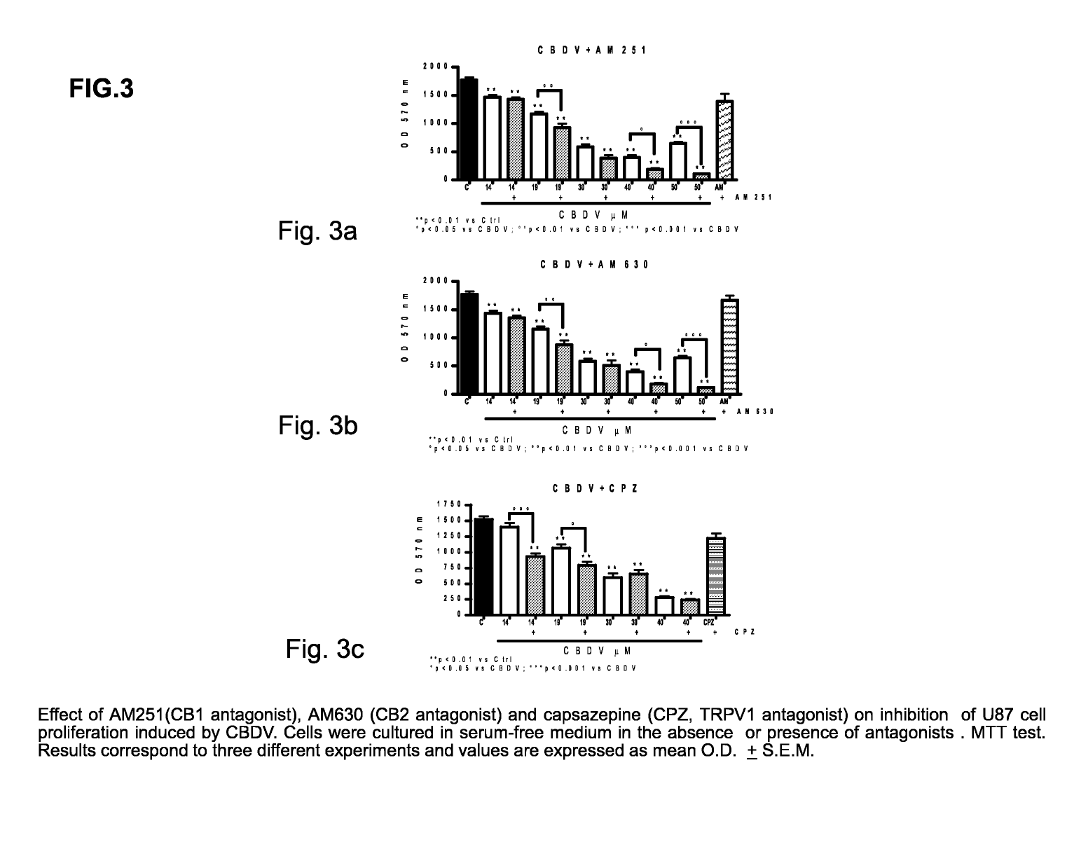

Similar experiments were carried out with CBDV (FIG. 3A-C). Surprisingly, both the CB1 cannabinoid receptor antagonist AM251 (0.5 μM) and CB2 cannabinoid receptor antagonist AM630 (0.5 μM), when added to CBDV-treated cells, were able to significantly increase the inhibitory action of CBDV on glioma cell growth at 19, 40, and 50 μM concentrations. When TRPV1 vanilloid receptor antagonist capsazepine (0.625 μM) was used, a significant increase of the inhibitory action of CBDV was seen only at 14 and 19 μM (FIG. 3C).

THCV:

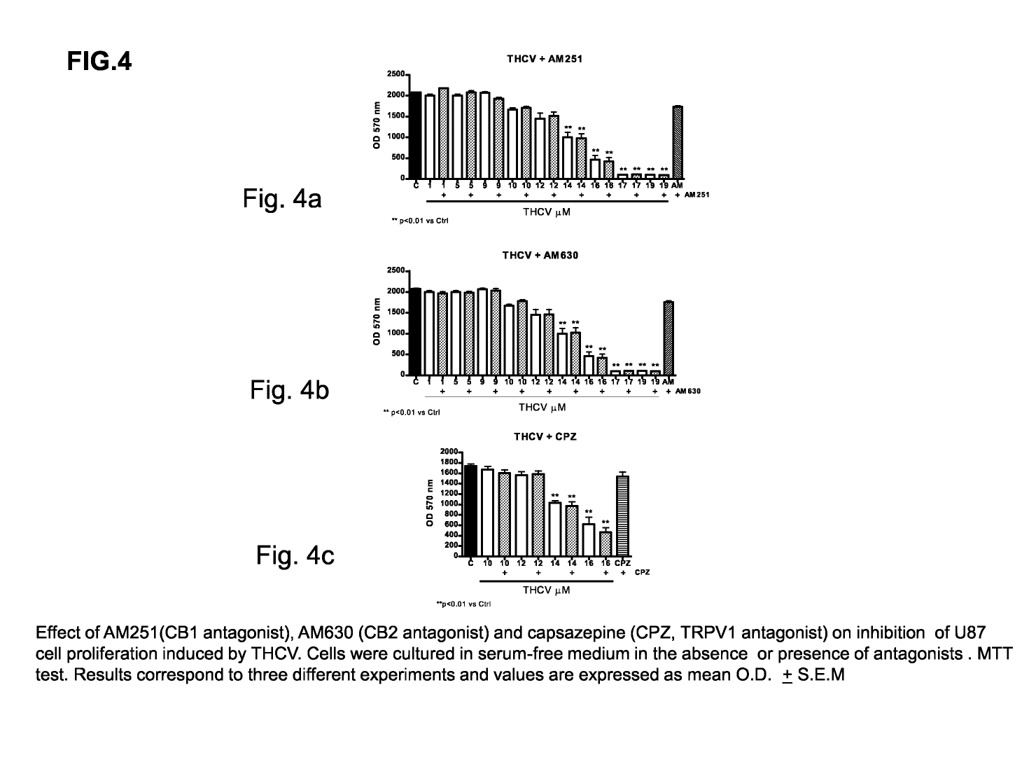

For THCV, none of the antagonists was effective in reversing and/or potentiating the effect of the phytocannabinoid at any of the tested concentrations (FIG. 4A-C).

In a further experiment THCV was evaluated in combination with CBD (CBD and THC both being compounds which have been shown to be effective against glioma alone and in combination.

### Example 5

### Evaluation of the Anti-Proliferative Effect Induced by the Association of CBD and THCV

This experiment was conducted due to the findings above that the different phytocannabinoids appear to act via different mechanisms and consequently may prove more beneficial when used in combinations. By way of example THCV was evaluated together with CBD. The two phytocannabinoids were evaluated using a combination of sub-effective concentrations to determine if there was any additive/synergistic effect in differing combinations (concentrations and ratios) on inhibition of proliferation.

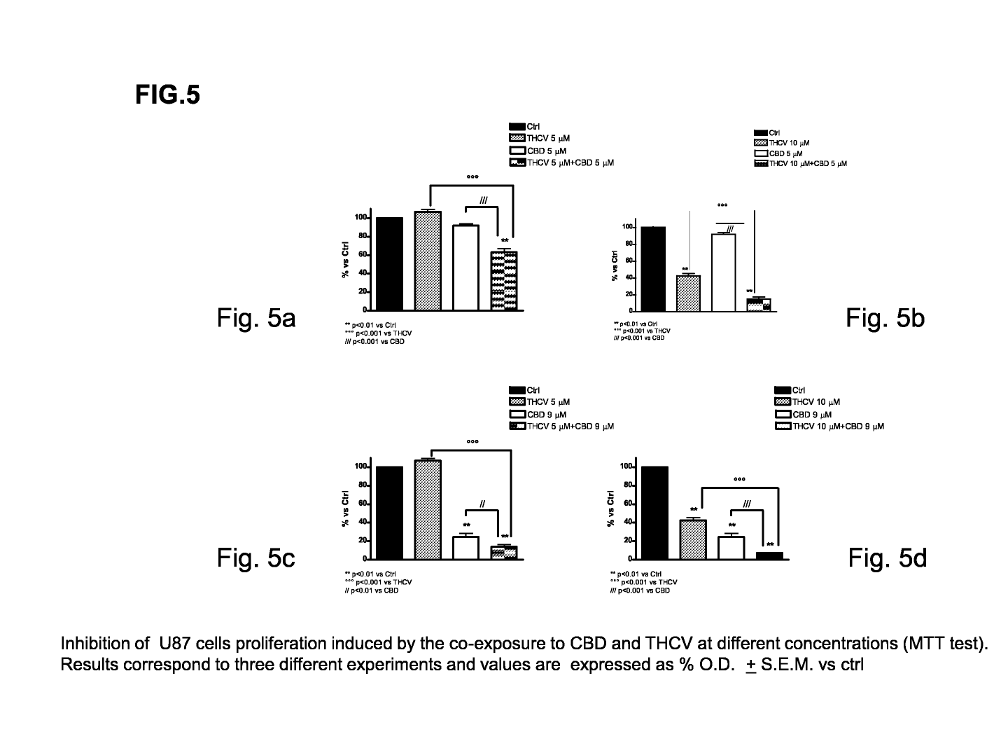

As shown in FIG. 5-A, the co-exposure of U87 cells with the two ineffective concentrations of THCV and CBD (5 μM+5 μM), caused a significant reduction in tumour cell viability (MTT test).

When the effective concentration of 10 μM THCV was used with the ineffective concentration of CBD (5 μM), a significant increase of the inhibitory properties of THCV (FIG. 5-B) was also observed.

In the same way, when the effective concentration of 9 μM CBD was used in combination with the ineffective concentration of THCV (5 μM), an increase of the inhibitory effect of CBD on tumour cells viability (FIG. 5-C) was also observed.

Finally, when an effective concentrations of the two phytocannabinoids (10 and 9 μM, respectively), was used in combination, a further significant decrease of cell viability (FIG. 5-D) was also observed.

This example provides a basis for looking at other combinations of phytocannabinoids in both effective and sub effective doses (of an individual cannabinoid).

### Example 6

### Evaluation of the Apoptotic Effect Induced by the Association of CBD and THCV

To better understand the cellular mechanism underlying the “potentiation” of the inhibitory effect on cell growth obtained with the combination of CBD and THCV, a set of experiments aimed at evaluating the presence of apoptosis induced by the two drugs alone or in combination was undertaken.

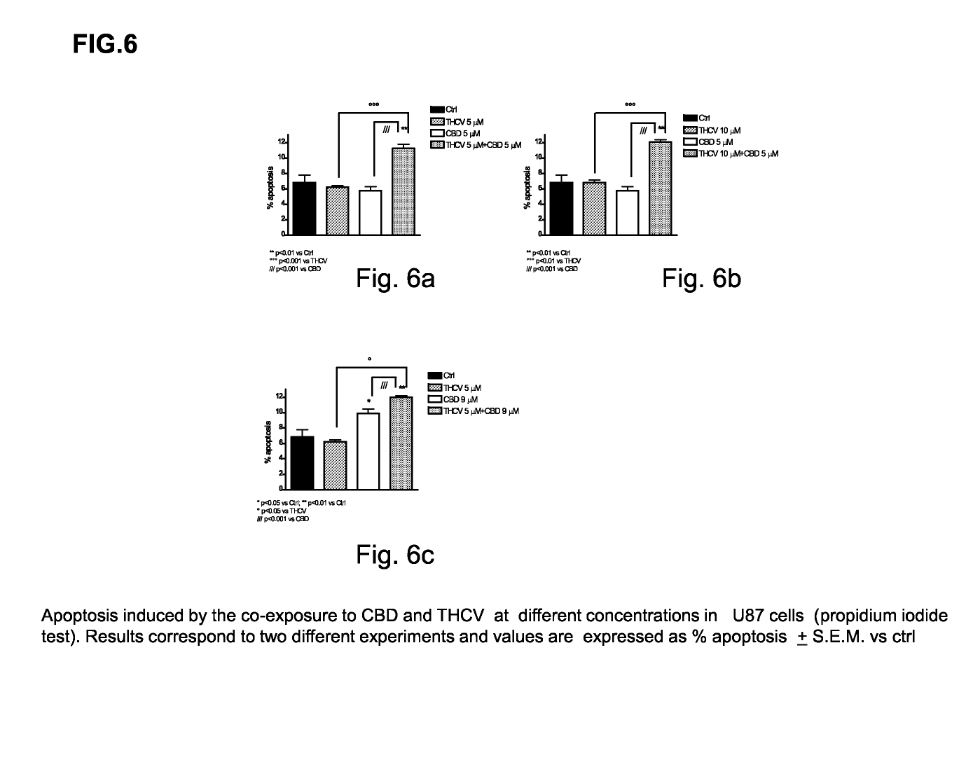

As shown in FIGS. 6.A-B, the exposure of U87 cells to the concentrations of THCV (5 and 10 μM) and CBD (5 μM) alone did not cause any apoptosis in U87 cells. In contrast, CBD at 9 μM caused a significant induction of apoptosis (FIG. 6-C).

When the non-apoptotic concentration of 5 μM or 10 μM THCV were used in combination with the non-apoptotic concentration of CBD (5 μM), a significant increase of apoptosis (FIGS. 6.A-B) was observed.

When the non-apoptotic concentration of 5 μM THCV was co-administered to the cells with the Pro-apoptotic concentration of CBD (9 μM), a significant increase of apoptosis (FIG. 6-C) was observed.

### Example 7

### Evaluation of the Anti-Migratory and Anti-Invasive Effects of THCV and CBDV

THCV:

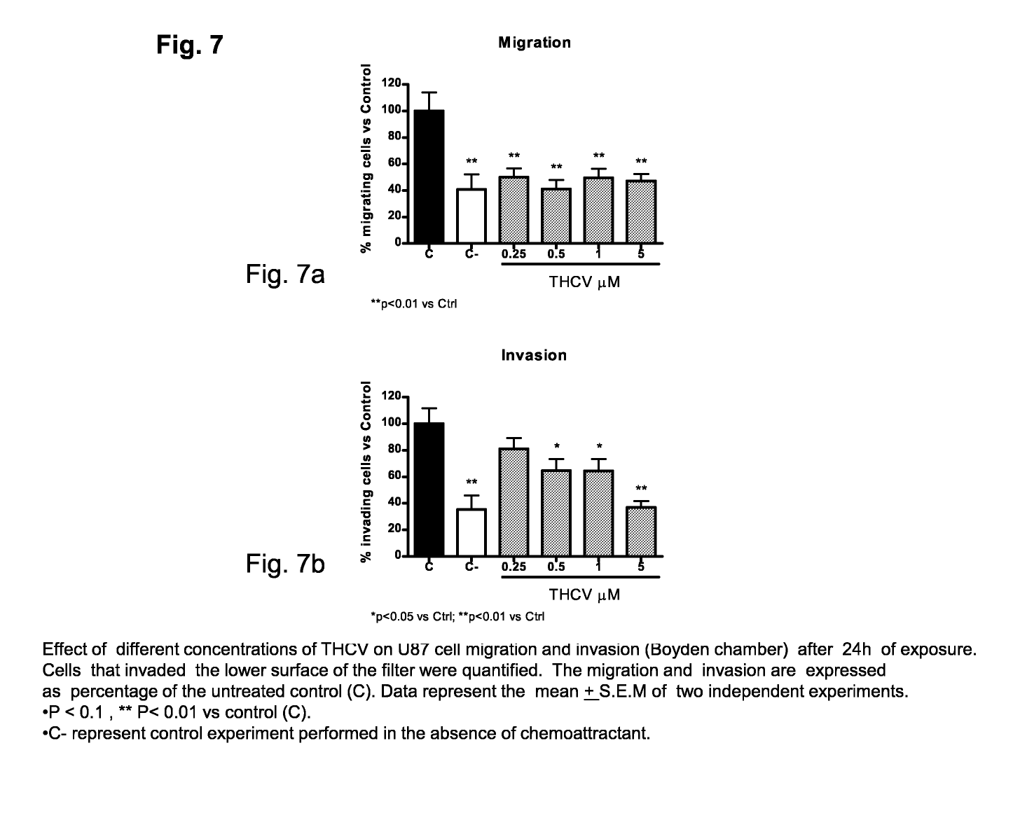

The effect of THCV on U87 glioma cells invasion and motility was determined by Boyden chamber assay. THCV significantly inhibited, by 55%, the migration of the cells through the gelatine-coated filters, irrespectively of the concentrations used (FIG. 7-A). The effect was even evident at concentrations as low as 0.25 μM.

Previous experiments addressing the impact of THCV on cellular viability demonstrated that concentrations effective in inhibiting cell motility were very far from those causing inhibition of cell viability (FIG. 1, MTT test, IC50 13.8 μM±1).

The matrigel invasion assay was carried out to further examine the effect of THCV on the invasiveness on U87 glioma cells. As shown in FIG. 7-B, THCV treatment caused a significant inhibition of cell invasiveness, about 35%, at concentrations as low as 0.5 and 1 μM, and of 55% at 5 μM.

CBDV:

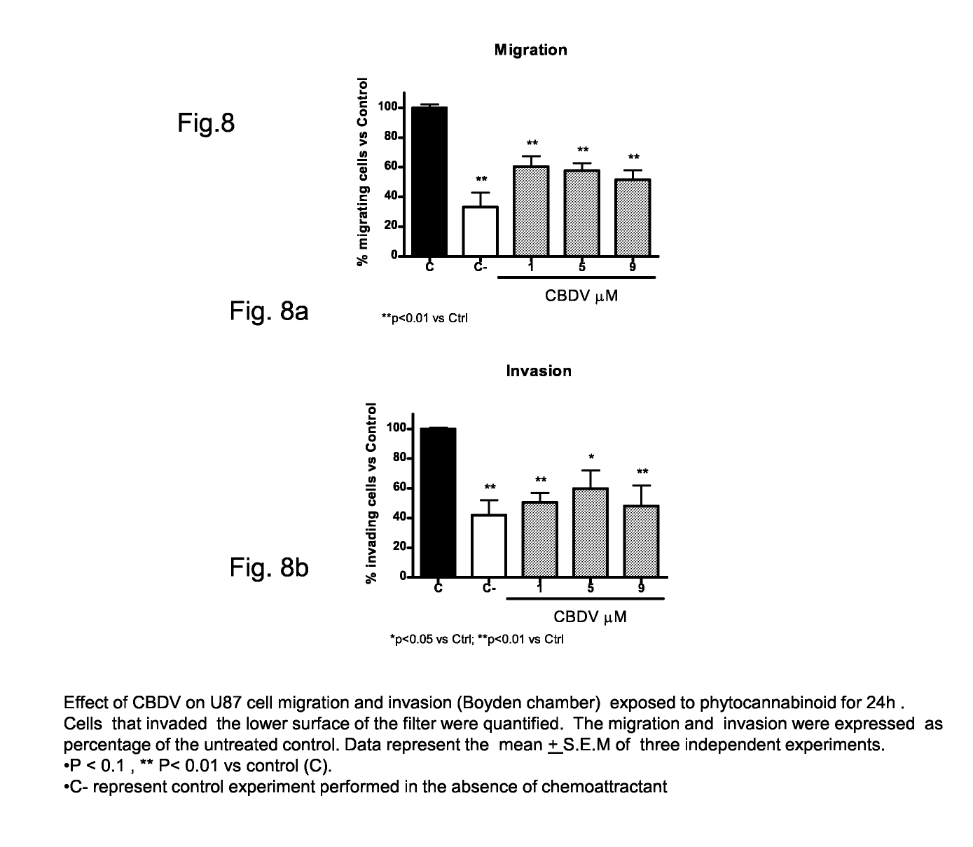

CBDV significantly inhibited the migration of the cells through the gelatine-coated filters (FIG. 8-A). The effect was evident at concentrations as low as 1 μM.

Previous experiments demonstrated that concentrations effective in inhibiting cell motility were far from those causing inhibition of cell viability (FIG. 1, MTT test, IC50 24.17 μM±1.02).

The matrigel invasion assay used to test invasiveness showed that the number of U87 cells able to invade through the chambers was significantly decreased by exposure to CBDV with an average effect of 45% already evident at concentrations as low as 1 μM (FIG. 8-B).

### Conclusions from Examples 1-7.

The results from Examples 1-7 show that CBG, CBDV and THCV all induce an inhibitory effect on U87 cells growth curves. However, the different shape of the dose-effect curves suggests that the three phytocannabinoids possess different mechanisms of action and thus may benefit in being used in combination with one another or with other cannabinoids such as THC and/or CBD which have also been shown to exhibit synergism in glioma and breast cancer cell lines.

THCV shows a very steep dose-response curve, between 12 and 19 μM, consistent with an “all or nothing” response suggesting a “non-receptor-mediated response”.

The results also established the involvement of cannabinoids/vanilloid receptors in some of the phytocannabinoid effects. Cannabinoids and vanilloid receptor antagonists evoked very different responses depending on the phytocannabinoids examined. The partial ability of AM251 to antagonize CBG suggests the involvement of CB1 receptor although the antagonist was not effective at all the CBG concentrations used.

In contrast, both CB1 and CB2 antagonists seem to increase the inhibitory effect of CBDV at all the tested concentrations, except for CBDV 14 and 30 μM.

The CBDV inhibition of cells growth was also enhanced by capsazepine but restricted only to the lower concentrations (14 and 19 μM).

These data seem to suggest that in tumour glioma cells, the pharmacological blockade of endocannabinoid and/or endovanilloid systems can favour the anti-proliferative effects of CBDV.

Finally, THCV effects were not antagonized by CB1, CB2 and TRPV1 antagonists, suggesting a cannabinoid- and/or vanilloid-receptors independent mechanism.

The association of CBD with THCV produced two positive effects:
1. The co-exposure of the tumour cells to concentrations of phytocannabinoids per se not inducing any effect on cells growth resulted in a significant inhibition of their viability. This was strengthened by the increase in apoptotic cells following the drug association;
2. Interestingly, the ineffective doses of CBD and/or THCV can enhance the efficacy of the active dose of each drug, in MTT assay as well as in apoptotic studies.

Thus, the obtained results indicate that combined treatment of THCV and CBD can result in an additive/synergistic effect in inhibiting tumour cells proliferation.

Moreover, the results provide evidence for the first time of the ability of THCV and CBDV to inhibit the migratory/invasive feature of glioma cells. This represents a very positive result because of the fundamental role of these processes in the aggressive behaviour of gliomas. These effects were already very significant at concentrations lower than those required to inhibit cell proliferation.

In a further set of experiments, Examples 8-10 the compounds were evaluated on a different cell line, T98G, which was obtained from the American Type Culture Collection (Rockville, USA). The protocols employed were as described above.

### Example 8

### Evaluation of the Anti-Proliferative Effect of CBDV, CBG and THCV in a T98G Cell Line

MTT Test:

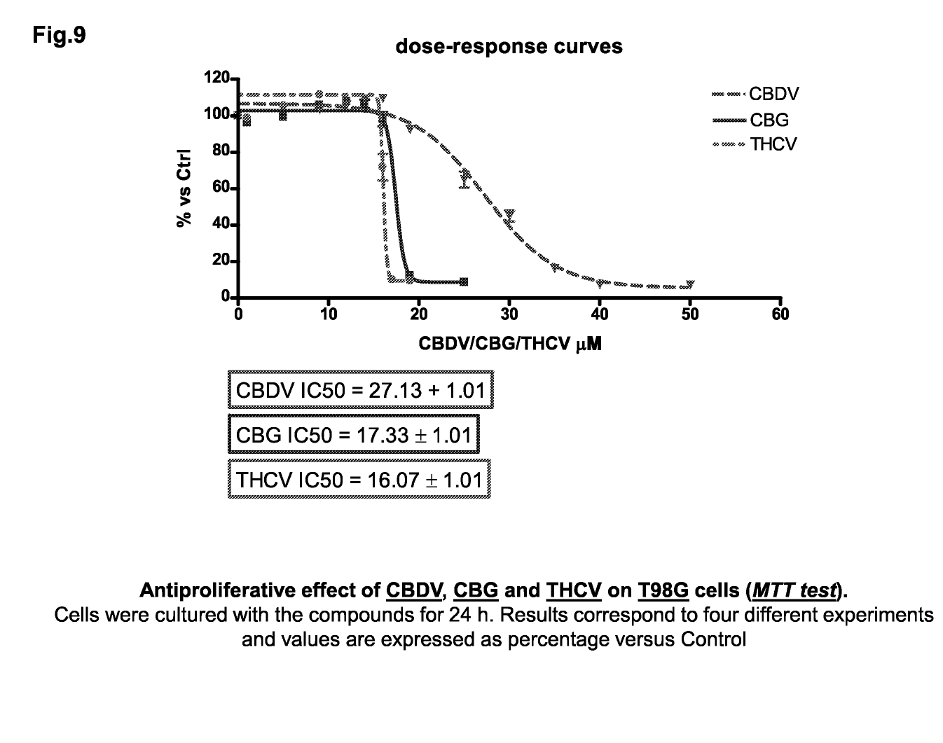

The addition of CBDV, CBG and THCV to the culture medium led to a dramatic drop of mitochondrial oxidative metabolism (MTT test), in a concentration-dependent manner, already evident 24 h after cannabinoid exposure with an IC50 of 27.13, 17.33 and 16.07 μM, respectively (FIG. 9).

Trypan Blue Test:

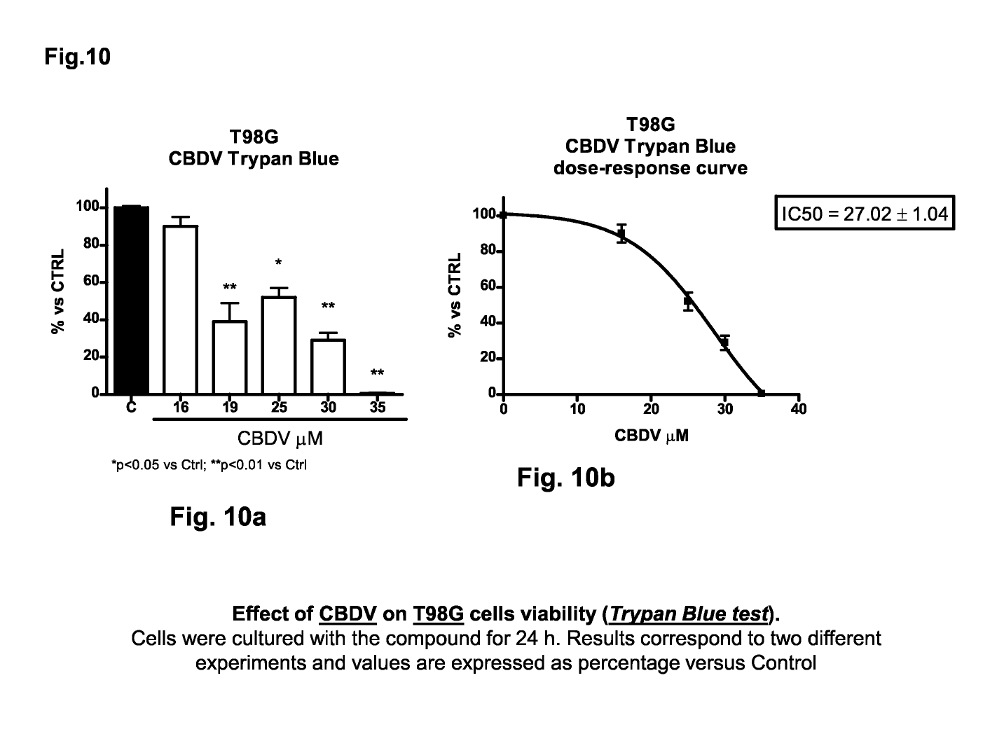

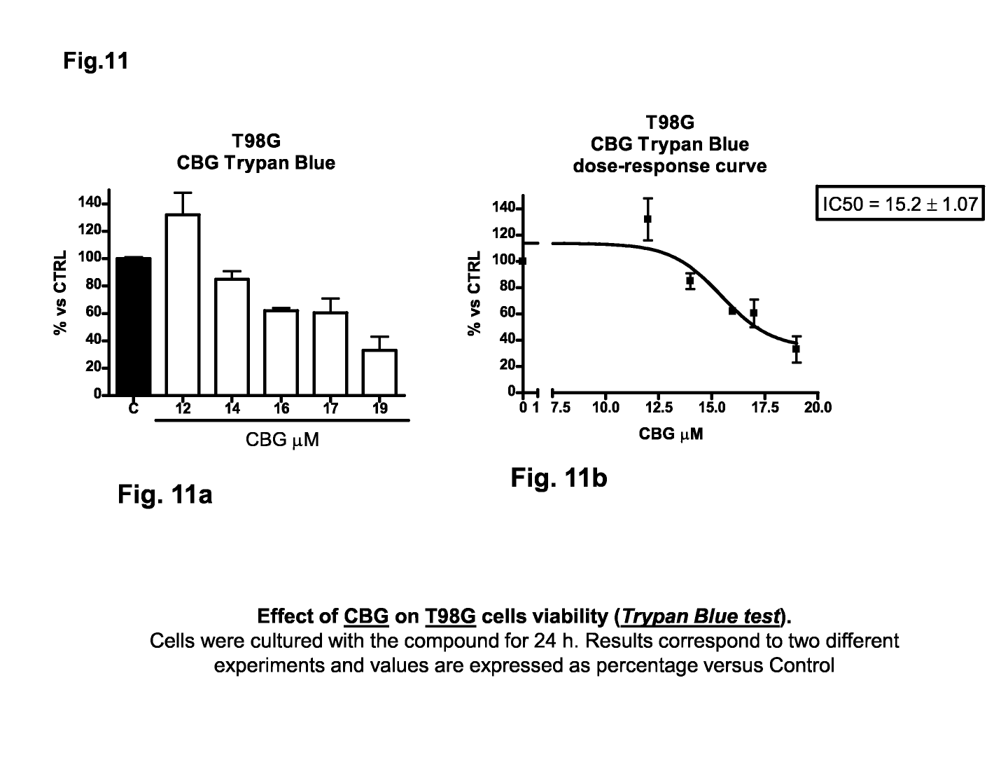

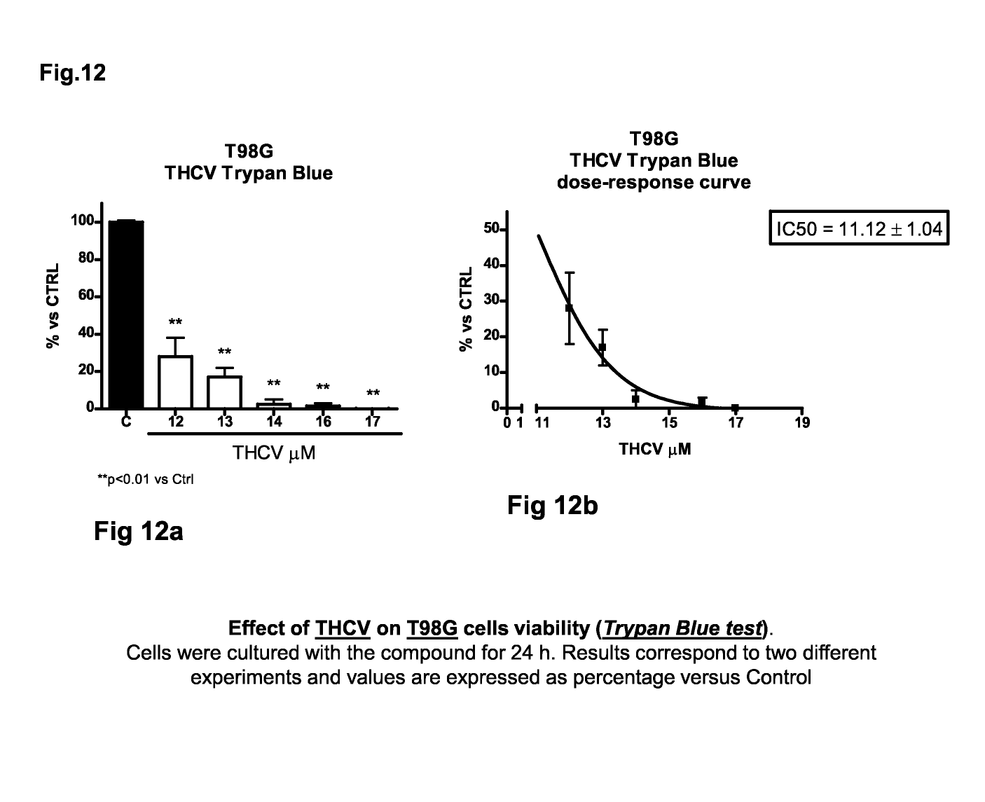

To further confirm the ability of CBDV, CBG and THCV to inhibit T98G cell growth, a Trypan Blue test was also conducted. As shown in FIGS. 10A and 10B, 11A and 11B and 12A and B, CBDV, CBG and THCV all inhibited cells viability in the same dose range as for MTT test.

### Example 9

### Evaluation of the Involvement of Cannabinoid Receptors in the Anti-Proliferative Effect of CBDV, CBG and THCV in a T98G Cell Line

Further experiments aimed at clarifying the role of cannabinoid receptors in the anti-proliferative effect of CBDV, CBG and THCV were conducted.

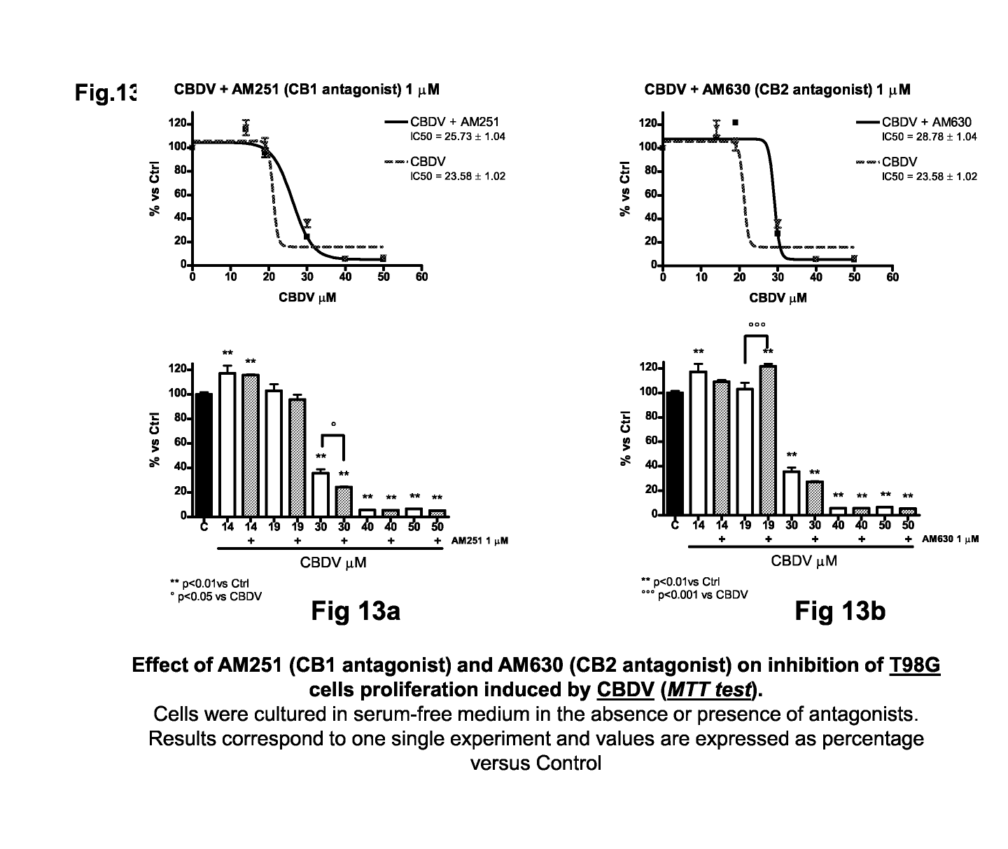

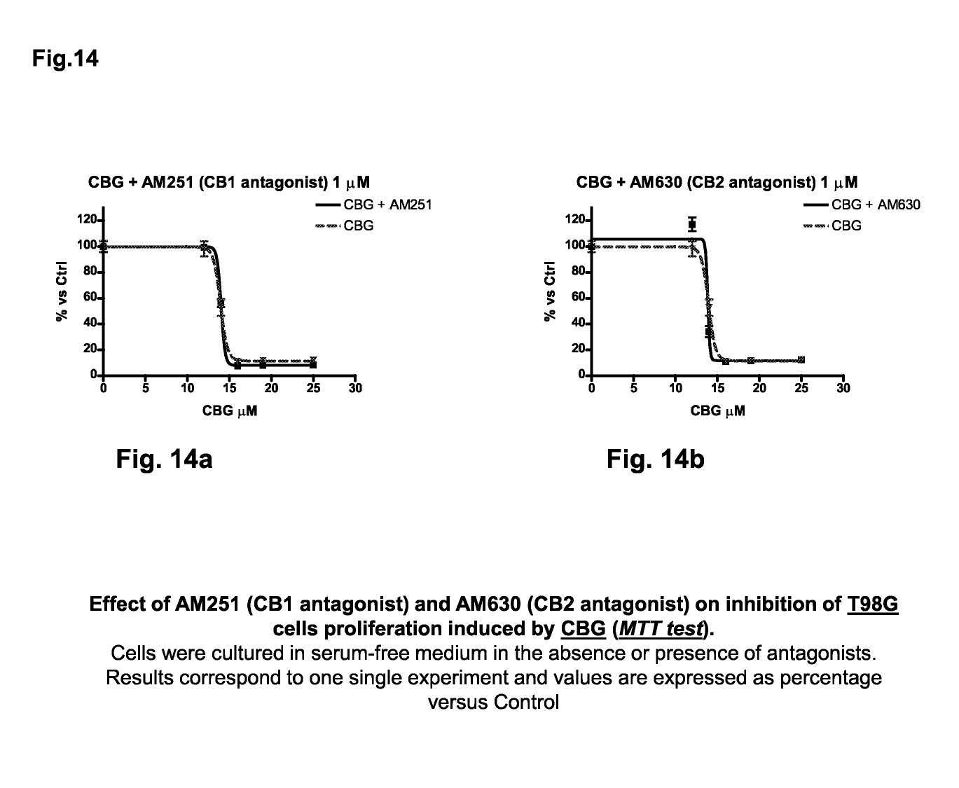

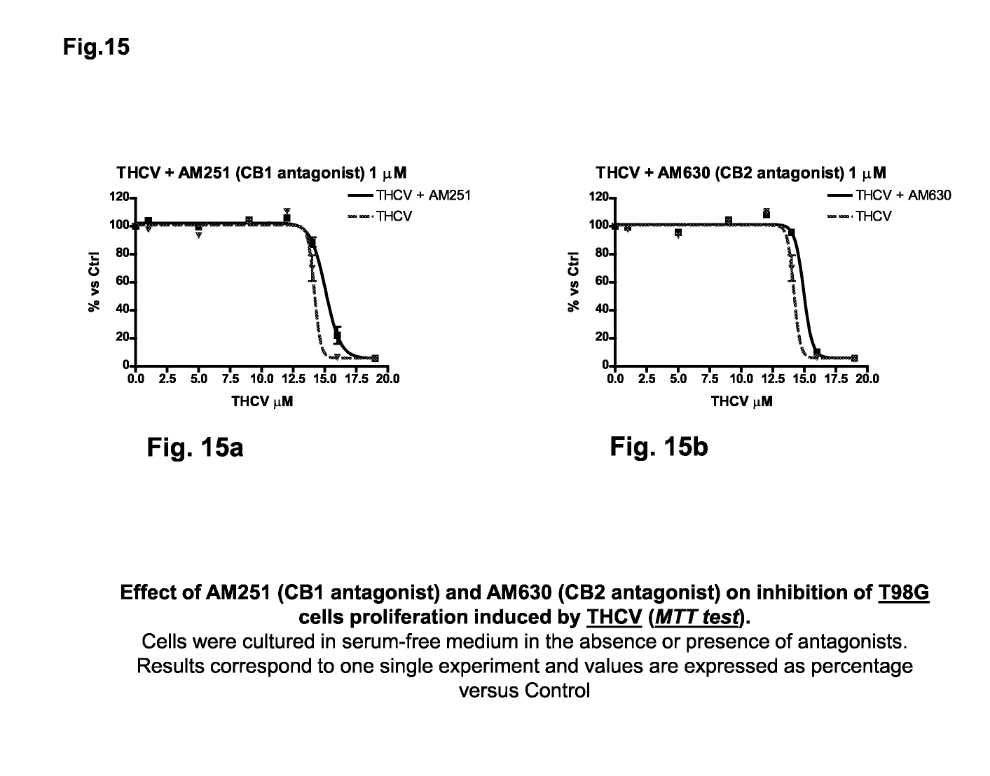

Of three compounds only CBDV exhibited some sensitivity to the pre-treatment with AM251 (CB1 antagonist) and AM630 (CB2 antagonist) (FIGS. 13A and 13B), whereas the anti-proliferative effect of CBG and THCV was unaffected by the pre-treatment with these antagonists (FIGS. 14a and 14B, FIGS. 15A and 15B)

### Example 10

### Evaluation of the Anti-Migratory and Anti-Invasive Effects of CBDV, CBG and THCV in T98G Cell Line

CBDV:

The effect of CBDV on T98G glioma cells invasion and motility was determined by Boyden chamber assay.

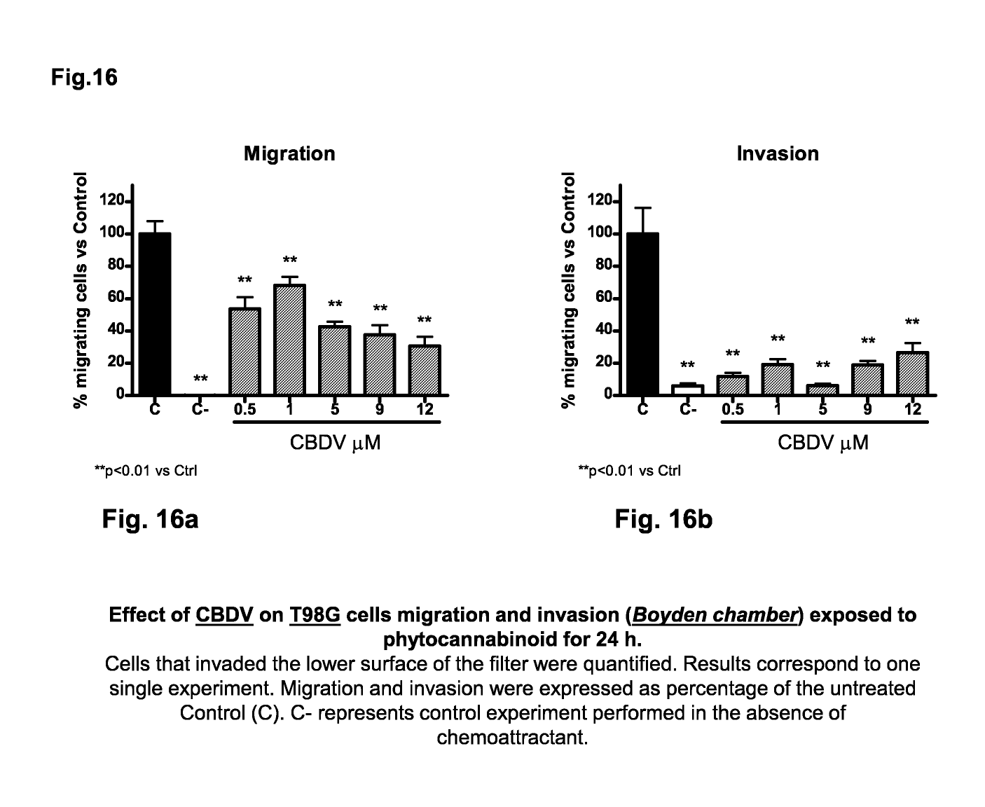

CBDV significantly inhibited, by 55%, the migration of the cells through the filters, irrespectively of the concentrations used (FIG. 16A). The effect was even evident at concentrations as low as 0.5 μM. The concentrations effective in inhibiting cell motility were very far from those causing inhibition of cell viability (Contrast with FIG. 9, MTT test, IC50 27 μM±1).

The matrigel invasion assay was carried out to further examine the effect of the CBDV on the invasiveness of T98G glioma cells. As shown in FIG. 16B, CBDV treatment caused a significant inhibition of cell invasiveness of about 70% in a concentration range from 0.5 to 12 μM.

CBG:

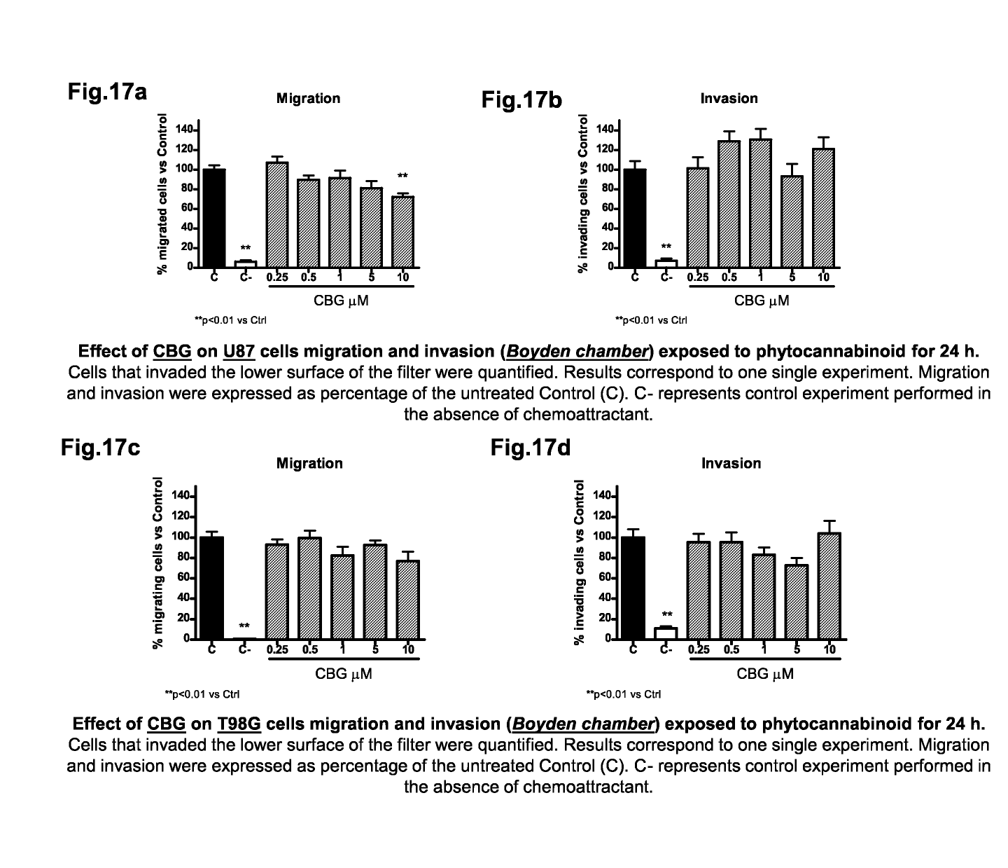

The effect of CBG on migration and invasion was tested both in U87 and in T98G cells lines. CBG did not inhibit either the migration or the invasion of the two different glioma cell lines at the tested concentrations (FIGS. 17A to 17D).

THCV:

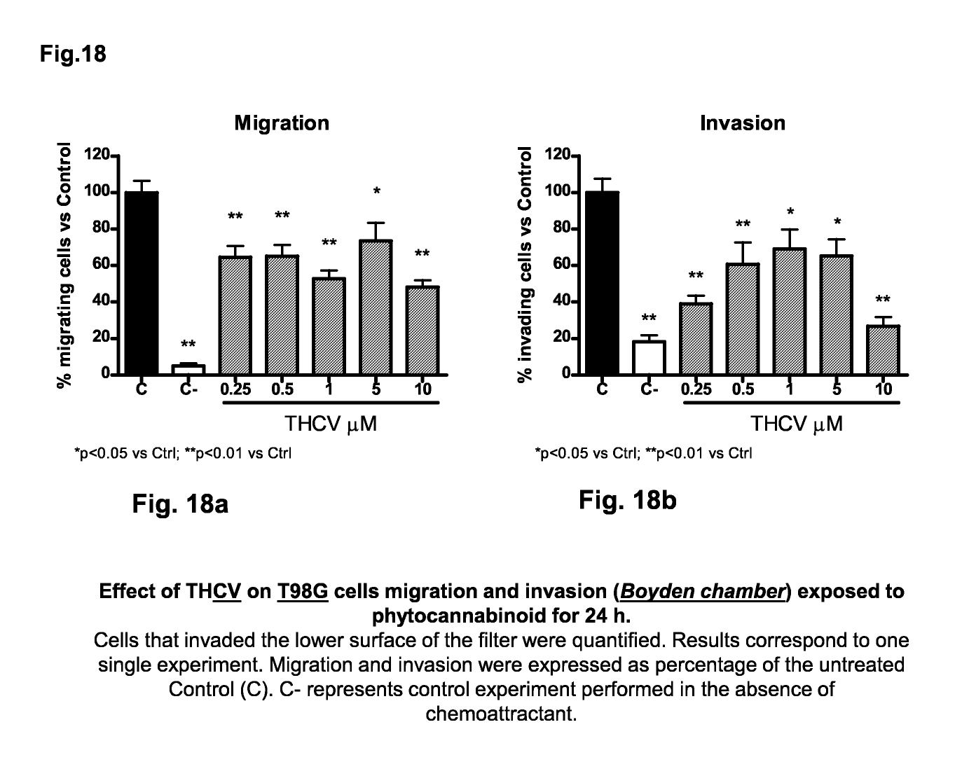

The effect of THCV on T98G cells migration (FIG. 18A) and invasion (FIG. 18B) was lower than that shown with CBDV. Migration was reduced by about 50% independently from the used concentration and when invasion was considered THCV showed a U-shaped dose-response curve (FIG. 18B).

The results reported here demonstrate that CBDV, CBG and THCV all induce an inhibitory effect on T98G cells growth curves. THCV and CBG showed a very steep dose-response curve, being the range of inhibition of cells growth between 10 and 20 μM, consistent with an “all or nothing” response.

In contrast CBDV appears less potent, but the shape of its dose-response curve is more consistent with a receptor-mediated hypothesis. The inhibition of T98G cells growth was obtained in the same dose range used for U87, confirming that phytocannabinoids affect the two different glioma lines with the same potency.

Applicant also established that the effects were not significantly antagonized by AM251 or AM630, suggesting the presence of a cannabinoid-receptor independent mechanism.

The results provide evidence of the ability of CBDV and THCV to inhibit the migratory/invasive feature of glioma cell. This represents a very positive result because of the fundamental role of these processes in the aggressive behaviour of gliomas. These effects were very significant at concentrations lower than those required to inhibit cells proliferation.

In contrast to THCV and CBDV, CBG did not share this property and did not affect cells migration and invasion either in U87 or in T98G cells.

### CONCLUSION

THCV and CBDV demonstrate anti-proliferative and anti-migratory/ant-invasive effects on glioma cells at concentrations where anti-proliferative effects are not seen.

### Example 11

### Evaluation of the Effect of CBDA and THCA on Migration in Breast (MDA-MB 231)

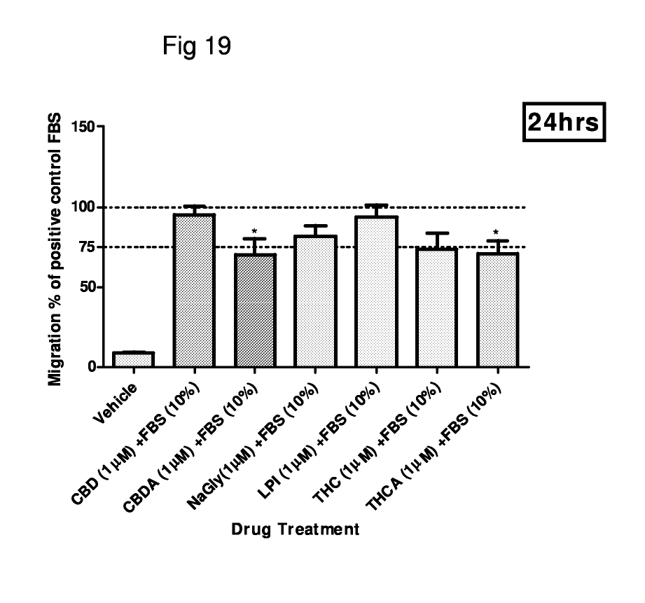

Whilst it is known that the phytocannabinoid acids of THCA and CBDA are anti-proliferative the applicant has also demonstrated that they inhibit migration in a statistically significant manner in a breast cell line as illustrated in FIG. 19.

1. THCV, CBDV, CBDA or THCA for use in the treatment of an invasive or migratory cancer.

2. THCV, CBDV, CBDA or THCA as claimed in claim 1 for use in the treatment of a glioma or breast cancer.

3. THCV or CBDV as claimed in claim 1 or 2 for use in the treatment of GBM.

4. THCA or CBDA as claimed in claim 1 or 2 for use in the treatment of breast cancer.

5. THCV as claimed in any of claims 1 to 3 wherein the THCV is isolated THCV.

6. THCV as claimed in any of claims 1 to 3 wherein the THCV is synthetic.

7. THCV as claimed in any of claims 1 to 3 wherein the THCV is present in a plant extract.

8. THCV as claimed in claim 7 wherein the THCV is the primary cannabinoid in the plant extract.

9. THCV as claimed in claim 8 wherein the THCV comprises at least 50% by weight of the total weight of cannabinoids.

10. THCV as claimed in claim 8 wherein the THCV comprises at least 50% by weight of the plant extract.

11. CBDV as claimed in any of claims 1 to 3 wherein the CBDV is isolated CBDV.

12. CBDV as claimed in any of claims 1 to 3 wherein the CBDV is synthetic.

13. CBDV as claimed in any of claims 1 to 3 wherein the CBDV is present in a plant extract.

14. CBDV as claimed in claim 13 wherein the CBDV is the primary cannabinoid in the plant extract.

15. CBDV as claimed in claim 14 wherein the CBDV comprises at least 50% by weight of the total weight of cannabinoids.

16. CBDV as claimed in claim 14 wherein the CBDV comprises at least 50% by weight of the plant extract.

17. THCA as claimed in any of claim 1 to 2 or 4 wherein the THCA is isolated THCA.

18. THCA as claimed in any of claim 1 to 2 or 4 wherein the THCA is synthetic.

19. THCA as claimed in any of claim 1 to 2 or 4 wherein the THCA is present in a plant extract.

20. THCA as claimed in claim 17 wherein the THCA is the primary cannabinoid in the plant extract.

21. THCA as claimed in claim 18 wherein the THCA comprises at least 50% by weight of the total weight of cannabinoids.

22. THCA as claimed in claim 18 wherein the THCA comprises at least 50% by weight of the plant extract.

23. CBDA as claimed in any of claim 1 to 2 or 4 wherein the CBDA is isolated CBDA.

24. CBDA as claimed in any of claim 1 to 2 or 4 wherein the CBDA is synthetic.

25. CBDA as claimed in any of claim 1 to 2 or 4 wherein the CBDA is present in a plant extract.

26. CBDA as claimed in claim 25 wherein the CBDA is the primary cannabinoid in the plant extract.

27. CBDA as claimed in claim 26 wherein the CBDA comprises at least 50% by weight of the total weight of cannabinoids.

28. CBDA as claimed in claim 26 wherein the CBDA comprises at least 50% by weight of the plant extract.

29. THCV, CBDV, CBDA or THCA as claimed in any of the preceding claims for use in combination with one or more other phytocannabinoids.

30. THCV, CBDV, CBDA or THCA as claimed in claim 29 wherein the one or more other phytocannabinoids are selected from the group comprising each other and additionally one or more of: THC, CBD, CBG, CBGA, CBDVA and THCVA.

31. The combination of THCV and CBD for use in the treatment of an invasive or migratory cancer.

32. THCV, CBDV, CBDA or THCA as claimed in any of the preceding claims for use to inhibit one or more of: proliferation, invasiveness or migration.
